(19) United States
(12) Patent Application Publication
(10) Pub. No.: US 2010/0099979 A1
(43) Pub. Date: Apr. 22, 2010

(54) SPATIAL CHARACTERIZATION OF A STRUCTURE LOCATED WITHIN AN OBJECT BY IDENTIFYING 2D REPRESENTATIONS OF THE STRUCTURE WITHIN SECTION PLANES

(76) Inventors: Gert Schoonenberg, Eindhoven (NL); Onno Wink, Eindhoven (NL); Babak Movassaghi, Eindhoven (NL)

Correspondence Address: PHILIPS INTELLECTUAL PROPERTY & STANDARDS, P.O. BOX 3001, BRIARCLIFF MANOR, NY, 10510, US

(21) Appl. No.: 12/444,057

(22) PCT Filed: Sep. 20, 2007
(86) PCT No.: PCT/IB07/53828

§ 371 Date: Apr. 2, 2009

(51) Int. Cl. A61B 5/05; G06K 9/62; G01N 23/04

(52) U.S. Cl. 600/424; 382/132; 378/62

(57) ABSTRACT

It is described a virtual pullback as a visualization and quantification tool that allows an interventional cardiologist to easily assess stent expansion. The virtual pullback visualizes the stent and/or the vessel lumen similar to an Intravascular Ultrasound (IVUS) pullback. The virtual pullback is performed in volumetric data along a reference line. The volumetric data can be a reconstruction of rotational 2D X-ray attenuation data. Planes perpendicular to the reference line are visualized as the position along the reference line changes. This view is for interventional cardiologists a very familiar view as they resemble IVUS data and may show a section plane through a vessel lumen or a stent. In these perpendicular section planes automatic measurements, such as minimum and maximum diameter, and cross sectional area of the stent can be calculated and displayed. Combining these 2D measurements allows also volumetric measurements to be calculated and displayed.

### FIELD OF INVENTION

The present invention generally relates to the field of digital image processing, in particular for medical purposes in order to provide for a visualization and for a quantitative analysis of an object being inserted within a body of a patient.

Specifically, the present invention relates to a method for spatially characterizing a structure located within an object under examination, in particular for spatially characterizing a medical device being inserted into the body of a patient.

Further, the present invention relates to a data processing device and to a medical X-ray examination apparatus, in particular a C-arm system or a computed tomography system, comprising the described data processing device, wherein the data processing device is adapted for spatially characterizing a structure located within an object under examination, in particular for spatially characterizing a medical device being inserted into the body of a patient.

Furthermore, the present invention relates to a computer-readable medium and to a program element having instructions for controlling the above-mentioned method for spatially characterizing a structure located within an object under examination, in particular for spatially characterizing a medical device being inserted into the body of a patient.

### ART BACKGROUND

During coronary interventions interventional cardiologists introduce a stent into a coronary vessel of a patient. Thereby, a stent delivery catheter is used. When the stent is positioned at the right position the interventional cardiologist expands the stent by increasing the pressure within a balloon being located inside the stent.

During such an intervention the stent expansion carried out in the inside of the patient's body cannot by observed directly. There are typically only available X-ray images or data from an intravascular device, e.g. an intravascular ultrasound (IVUS) device. However, determining and monitoring the stent expansion on images being provided by X-ray imaging and/or by IVUS is very difficult.

In order to increase the visibility of a stent being inserted into a patient's vessel Philips has developed a technique called “StentBoost”. Thereby, a StentBoost image is produced using radio opaque markers of a delivery balloon. The result is a still image of the stent with enhanced edges and the region of interest around it.

WO 2004/081877 A1 discloses an X-ray imaging method for forming a set of a plurality of two-dimensional X-Ray projection images of a medical or veterinary object to be examined through a scanning rotation by an X-Ray source viz-à-viz the object. Such X-Ray images are acquired at respective predetermined time instants with respect to a functionality process produced by the object. From said set of X-Ray projection images by back-projection a three-dimensional volume image of the object is reconstructed. In particular, an appropriate motion correction is derived for the respective two-dimensional images, and subsequently, as based on a motion vector field from the various corrected two-dimensional images, the intended three-dimensional volume of the object is reconstructed.

WO 99/13432 discloses an apparatus and a method for performing three-dimensional (3D) reconstructions of tortuous vessels such as coronary arteries. The reconstructions can be obtained by data fusion between biplane angiography and IVUS frames of a pullback sequence. The 3D course of the tortuous vessel is first determined from the angiograms and then combined with the two-dimensional (2D) representations regarding the 3D course using a data fusion apparatus and method. The determination of the 3D pullback path is represented by the external energy of the tortuous vessel and the internal energy of a line object such as a catheter.

There may be a need for providing for a detailed and precise spatial characterization of a structure located within an object.

### SUMMARY OF THE INVENTION

This need may be met by the subject matter according to the independent claims. Advantageous embodiments of the present invention are described by the dependent claims.

According to a first aspect of the invention there is provided a method for spatially characterizing a structure located within an object under examination, in particular for spatially characterizing a medical device being inserted into the body of a patient. The described method comprises the steps of (a) acquiring a volumetric dataset of the object under examination, (b) establishing a reference line within the volumetric dataset, (c) generating a plurality of section planes within the volumetric dataset, wherein the section planes are oriented at least approximately perpendicular to the reference line, and (d) identifying 2D representations of the structure within the plurality of section planes.

This first aspect of the invention is based on the idea that a certain structure within an object under examination can be spatially characterized in a manner, which is similar to the spatial information being provided by intravascular ultrasound (IVUS). In IVUS a small ultrasound (US) transducer in inserted by means of a catheter within the vessel structure of a patient representing the object under examination. By carrying out the method described herein a physician can be provided with the same type of information, which he is used to if he is experienced with IVUS. However, compared to IVUS the described method is much more convenient both for the physician and a patient because an elaborate pullback of an IVUS transducer being mounted to a catheter device within a vessel is no more necessary.

The volumetric dataset may be acquired by means of different examination procedures such as magnetic resonance tomography, positron emission tomography or single photon computed tomography. However, also other 3D imaging modalities may be used.

The section planes, which may also be denoted as cut planes, represent slices respectively slabs of the object under examination. The thickness of the slabs determines the spatial resolution of the described method in a direction aligned in parallel with the reference line.

The identification of the structure within the section plane may be carried out by applying known methods for image processing. Such methods are for instance based on thresholding, edge detection or region based for the segmentation or classification of said structure. Such a method is for instance the detection of edges or spatial transitions between regions within a section plane, which regions have a different brightness. The identification of the structure can also be performed using the whole volumetric dataset. Other known methods for image processing, such as segmentation methods, may be carried out for the identification of the structure in 3D.

It has to be pointed out that of course one or more of the identified 2D representation can be displayed by means of e.g. a monitor and a printer.

According to an embodiment of the invention the volumetric dataset represents the X-ray attenuation behavior of the object under examination. The volumetric dataset can be obtained in particular by means of an X-ray imaging device having an X-ray scanner rotating around the object under examination or at least around a region of interest within the object under examination. The X-ray scanner typically comprises an X-ray source and an X-ray detector arranged vis-à-vis each other. The X-ray imaging device may be for instance a computed tomography (CT) scanner or a C-arm system, which both allow for a sequential acquisition of two-dimensional (2D) projection data of the object under examination at different viewing angles. An appropriate 3D image reconstruction of the object under examination may be carried out by applying known reconstruction procedures such as filtered back projection or the like.

Preferably, the reference line runs in a three-dimensional (3D) volume within the object under examination in such a manner, that the reference line is aligned with the structure, which is supposed to be characterized. Thereby, the reference line may be located in an at least partially symmetric manner with respect to the structure.

According to a further embodiment of the invention the volumetric dataset is a motion compensated dataset. This may provide the advantage that also moving objects like e.g. the human heart can be investigated in an effective manner. Thereby, the number of 2D projection datasets, which have been acquired at various projection angles and which can be used for the 3D reconstruction, can be increased significantly. This is based on the fact that the time window within a movement of the object, in which datasets being usable for a 3D reconstruction of the object under examination can be acquired, can be elongated. In other words, for the reconstruction not only projection data showing the object under examination at a definite positional state but also projection data showing the object at different positional states can be used for the 3D image reconstruction. Thereby, depending on the required spatial precision projection data being assigned to more or less similar positional states may be used.

The motion compensation may be carried out by acquiring rotational projection data of an object under examination e.g. by employing a C-arm system. Thereby, the object under examination is equipped with reference markers being located e.g. on a guide wire. In the 2D projection images the markers on the guide wire and the guide wire itself, or markers on the medical device, e.g. a stent, itself and the guide wire, are automatically detected and used to correct the images for a motion of the object. After a motion correction of at least some of the projection datasets a 3D reconstruction is performed in a known manner. Thereby, the motion compensated volumetric dataset is generated. For more details regarding the generating of a motion compensated volumetric dataset reference is made to the international patent application WO 2004/081877 A1.

According to a further embodiment of the invention the reference line is defined by at least two reference markers being inserted into the object under examination. This may provide the advantage that in particular when a motion compensated volumetric dataset is used as the starting point for carrying out the described method, the volumetric dataset will be automatically centered with respect to a line being defined by the at least two reference markers.

In particular the reference markers can be inserted into a vessel of a patient under examination by employing a catheter device.

According to a further embodiment of the invention the reference line is defined by a guide wire. Thereby, the guide wire may be equipped with a plurality of reference markers. Using a guide wire representing a plurality of reference markers provides the further advantage that the reference line may be defined very precisely with respect the structure, which is supposed to be spatially characterized. In particular using a guide wire is advantageous for spatially characterizing a structure, which structure comprises not a straight but a rather complex line being formed within the 3D space of the object under examination.

Of course, reference markers are structures, which can be clearly seen on each 2D X-ray projection image such that a 3D reconstruction may be carried out even if the object under examination is moving in between different 2D data acquisitions obtained at different viewing angles. Thereby, the above described motion compensation may be used.

According to a further embodiment of the invention the method further comprises the step of visualizing the structure by displaying the identified 2D representations of the structure in a sequential manner. Preferably the sequence of the displayed 2D representations corresponds to a section plane, which is moving along the reference line. Thereby, every section plane represents a preferably perpendicular cross section of the structure.

At the beginning of the reference line a first section plane perpendicular to the reference line is defined or a slab of a certain thickness T is extracted and displayed. Along the reference line small steps of size S are made and each time a new section plane is displayed. This is done until the end of the reference line is reached.

This may provide the advantage that the spatial information regarding the structure, which information has been obtained in particular by means of X-radiation, is similar to the information, which can be obtained by means of intravascular ultrasound (IVUS). Thereby, a transducer being inserted into a vessel of the patient under examination is moved along the centerline of the vessel. In other words, the described method will allow for a visualization of the structure in an IVUS pullback like way. This virtual pullback is characterized by basically moving a section plane along the reference line that goes through the inside of the structure. The section planes being oriented perpendicular to the reference line are visualized as the position along the reference line changes. These views are in particular very familiar for the interventional cardiologists as they resemble IVUS data and show a section plane through a vessel. In case a stent is inserted into the vessel the stent can be visualized without having the hassles being related to known IVUS procedures.

It has to be mentioned that by displaying the various section planes in a sequential manner a pullback motion is mimicked rather than performed in reality. However, mimicking an IVUS acquisition will greatly assist the interventional cardiologist in assessing the stent deployment and expansion.

The user, e.g. the interventional cardiologist or the operator in the control room, can let the images be displayed continuously at a certain speed. Alternatively, the user can scroll through the different frames manually.

According to a further embodiment of the invention the further comprises the step of displaying a 3D model representation of the structure by combining a plurality of identified 2D representations being assigned to a plurality of different section planes. This may provide the advantage that a 3D model of the structure can be displayed in a projection view. Of course, the angle of the projection view can be varied manually or automatically such that a physician can get a realistic impressing of the spatial characteristics of the structure.

Further, a color-coding may be used in order to improve the 3D visualization of the structure. In case a stent is the structure, which is supposed to be characterized, a generic stent model can be used for an improved visualization.

According to a further embodiment of the invention the method further comprises the step of measuring at least one spatial dimension of at least some of the identified 2D representations. The measurement of spatial dimensions of the 2D representations of the structure may be carried out in an automated manner by applying known methods for image processing. An automatic measurement may be carried out e.g. by detecting a contour of the identified structure and after identifying such contour measuring for instance the maximum diameter or surface area inside the contour.

The described measurement may allow for a quantitative characterization of the structure, which makes the described method even more reliable, because a physician can be provided with absolute values regarding the size of the structure. Absolute values of the structure can be compared to e.g. standard values of a predefined range of values, which range is assigned to a known structure. This holds in particular for stents, which usually are specified with exact parameters regarding for instance the maximum allowable expansions.

Assessing not only a relative but also an absolute stent expansion may provide the advantage that the interventional cardiologist can be provided with not only qualitative but also with quantitative information about the expansion of a stent being inserted into a vessel in order to treat a stenosis.

The automated measurement and a subsequent calculation of spatial characteristics has the advantage that only a minimum user interaction is needed in order to carry out the described method and to provide a physician with valuable information during an interventional procedure.

It has to be mentioned that the described method can be carried out several times in sequence during a medical procedure where a stent is inserted in a predefined portion of a vessel and the stent is expanded in order to prevent for a further narrowing or even a closing of the vessel. This may allow for a precise monitoring of the actual stent expansion thus making the whole stent expansion procedure more secure.

According to a further embodiment of the invention the method further comprises the step of combining the at least one spatial dimension being measured for different 2D representations in such a manner that a 3D model representation of the structure is established. This may provide the advantage that also quantitative parameters regarding to the volume of the structure can be evaluated.

According to a further embodiment of the invention the spatial dimension is the diameter of the structure and/or the cross sectional area of the structure. This may provide the advantage that after having measured at least a plurality of different 2D representations being assigned to different section planes the minimum and the maximum diameter and/or the minimum and the maximum cross sectional area of the structure can be calculated very easily. However, these parameters may provide a physician with valuable information about an interventional procedure such as placing a stent within a stenosis of a patient's vessel system.

In case a stent is the structure to be characterized further very helpful parameters characterizing a stent placement procedure can be easily determined. Such parameters are e.g. the actual stent expansion relative to a maximal stent expansion or the actual stent expansion relative to the desired stent expansion. Thereby, the stent expansion can be characterized with reference to e.g. the diameter of the cross sectional surface size of the stent. Such automatic measurements will help the interventional cardiologist to easily and quickly access stent deployment and expansion. The corresponding dimensions can be manually selected by a user. The dimensions can be displayed or calculated fully automatically from the 2D slices being represented by the section planes and/or by a 3D volume of the structure being accessible by an appropriate combination of a plurality of the 2D slices, or by a 3D volume of the structure itself.

According to a further embodiment of the invention the method further comprises the step of displaying a diagram depicting the at least one spatial dimension as a function of the position of the corresponding section plane with respect to the reference line. In case the structure is a substantially cylindrical element such as a stent this may allow to plot the diameter of the cylindrical element versus the position along the center axis of the cylindrical element. Thereby, a quantitative information about the cylindrical element may be given in such a manner, which allows a physician to recognize the spatial characteristic of the structure very quickly and very easily.

Further, interventional cardiologists are used to interpret such kinds of diagrams from known quantitative coronary analysis (QCA) and/or from quantitative stent analysis procedures.

According to a further embodiment of the invention the reference line is located within a vessel lumen of a patient. This may provide the advantage that the described method may also be used for investigating at least certain regions of a vessel tree respectively a vascular system of a patient under examination. In order to provide a clear visualization of the vascular system it might be advantageous to use a contrast agent, which has been administered to the patient.

According to a further embodiment of the invention the structure is a predetermined region of a vessel tree. This may provide the advantage that not only elements, which have been inserted into the vascular system, but also the vascular system itself can be investigated in a precise manner. Thereby, a stenosis or any other narrowing of a vessel can be identified and spatially characterized.

According to a further embodiment of the invention at least one known property of a vessel lumen is used in order to identify the vessel lumen within the 2D representations. This may provide the advantage an automatic consistency check may be carried out whether it is really possible that the identified structure is a vessel lumen.

According to a further embodiment of the invention the structure is a stent. In this context a stent is an expandable wire form or perforated tube that is inserted into a vessel of a patient's body in order to prevent or counteract a disease-induced localized flow constriction. It has to be mentioned that also other elements may represent the described structure, which other elements can be inserted into the live human or animal body. For instance the structure can also be a stent graft device. A stent graft device is a tube composed of fabric supported by a metal mesh.

According to a further embodiment of the invention at least one known property of the stent or vessel lumen is used in order to identify the stent within the 2D representations. This may provide the advantage that a comparison of an identified structure within the measured lumen with e.g. the maximal possible expanded stent lumen may be carried out. Thereby, one may carry out an automatic consistency check whether it is really possible that the identified structure is an inserted stent. The reliability of a corresponding algorithm of such a consistency check will depend on such an input information.

According to a further embodiment of the invention the step of acquiring a volumetric dataset of the object under examination is carried out with contrast agent being inserted into the vessel lumen. This may provide the advantage that the morphology of at least a portion of the vascular system can be clearly visualized.

According to a further embodiment of the invention the method further comprises the step of acquiring a further volumetric dataset of the object under examination in the absence of a contrast agent. This may provide the advantage that both a first volumetric dataset in the presence and a further second volumetric dataset in the absence of contrast agent are available. Therefore, by using one and the same reference line two types of 2D representations can be identified. A first type of 2D representations shows predominantly the vessel lumen. The second type of 2D representations shows predominantly the stent being inserted into the vessel lumen. This may allow for a very precise comparison between the vessel lumen and the stent lumen. In particular, if the above-explained steps, which are related to quantitative measurements of the structure, are executed, a very precise quantitative comparison between the vessel lumen and the stent lumen can be accomplished. Thereby, the quantitative measurements may be carried out separately for both the vessel and the stent. Alternatively, the quantitative measurements may be carried out after the first type of 2D representations have been combined with the corresponding second type of 2D representations in order to generate a common display showing both the vessel and the stent in a clear manner.

According to a further aspect of the invention there is provided a data processing device for spatially characterizing a structure located within an object under examination, in particular for spatially characterizing a medical device being inserted into the body of a patient. The data processing device comprises (a) a data processor, which is adapted for performing exemplary embodiments of the above-described method and (b) a memory for storing the acquired volumetric dataset of the object under examination and/or for storing the identified 2D representation of the structure and/or for storing a movie of all or a selection of the identified 2D representations of the structure.

According to a further aspect of the invention there is provided a medical X-ray examination apparatus, in particular a C-arm system or a computed tomography system. The medical X-ray examination apparatus comprises the above-described data processing device.

According to a further aspect of the invention there is provided a computer-readable medium on which there is stored a computer program for spatially characterizing a structure located within an object under examination, in particular for spatially characterizing a medical device being inserted into the body of a patient. The computer program, when being executed by a data processor, is adapted for controlling exemplary embodiments of the above-described method.

According to a further aspect of the invention there is provided a program element for spatially characterizing a structure located within an object under examination, in particular for spatially characterizing a medical device being inserted into the body of a patient. The program element, when being executed by a data processor, is adapted for controlling exemplary embodiments of the above-described method.

The computer program element may be implemented as a computer readable instruction code in any suitable programming language, such as, for example, JAVA, C++, and may be stored on a computer-readable medium (removable disk, volatile or non-volatile memory, embedded memory/processor, etc.). The instruction code is operable to program a computer or other programmable device to carry out the intended functions. The computer program may be available from a network, such as the WorldWideWeb, from which it may be downloaded.

It has to be noted that embodiments of the invention have been described with reference to different subject matters. In particular, some embodiments have been described with reference to method type claims whereas other embodiments have been described with reference to apparatus type claims. However, a person skilled in the art will gather from the above and the following description that, unless other notified, in addition to any combination of features belonging to one type of subject matter also any combination between features relating to different subject matters, in particular between features of the method type claims and features of the apparatus type claims is considered to be disclosed with this application.

The aspects defined above and further aspects of the present invention are apparent from the examples of embodiment to be described hereinafter and are explained with reference to the examples of embodiment. The invention will be described in more detail hereinafter with reference to examples of embodiment but to which the invention is not limited.

### BRIEF DESCRIPTION OF THE DRAWINGS

FIG. 1a shows a schematic side view of a medical C-arm system.

FIG. 1b shows a perspective view of the X-ray swing arm shown in FIG. 1a.

FIG. 2 shows an illustration of two stents, one being in the unexpanded state and the other being in the expanded state.

FIG. 3 shows a flow chart on a method for spatially characterizing and visualizing a structure being located within an object under examination.

FIG. 4a shows an image depicting a volumetric representation of a guide wire and two stents.

FIG. 4b shows an image depicting a cross section of one of the stents shown in FIG. 4a in a perpendicular frame with respect to the longitudinal axis of the stent.

FIG. 4c shows an image depicting a perspective volumetric representation of contrast agent being inserted into a vessel lumen.

FIG. 5 shows a workflow diagram for controlling a proper stent deployment.

FIG. 6a shows a diagram depicting the stent diameter as a function of the longitudinal position within the stent.

FIG. 6b shows a diagram depicting the stent cross-sectional area as a function of the longitudinal position within the stent.

FIG. 7 shows a 3D representation of a deployed stent based on spatial measurements carried out by applying the described method.

FIG. 8 shows an data processing device for executing the preferred embodiment of the invention.

### DETAILED DESCRIPTION

The illustration in the drawing is schematically. It is noted that in different figures, similar or identical elements are provided with the same reference signs or with reference signs, which are different from the corresponding reference signs only within the first digit.

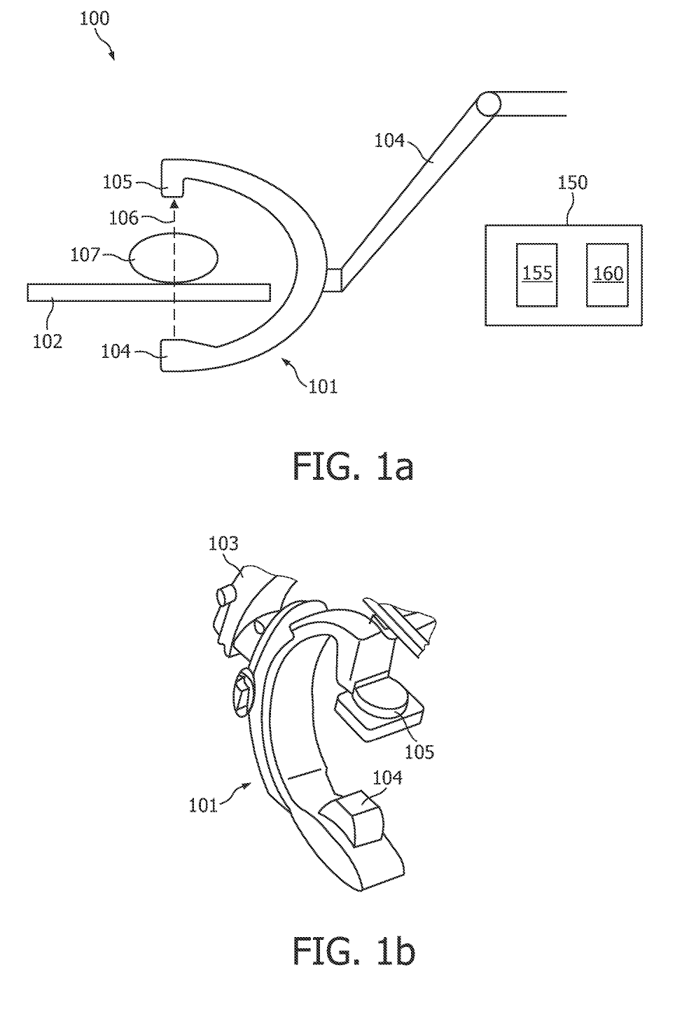

Referring to FIG. 1a and 1b of the drawing, a medical X-ray imaging system 100 according to an embodiment of the invention comprises a swing arm scanning system (C-arm) 101 supported proximal a patient table 102 by a robotic arm 103. Housed within the swing arm 101, there is provided an X-ray tube 104 and an X-ray detector 105. The X-ray detector 105 is arranged and configured to receive X-rays 106, which have passed through a patient 107 representing the object under examination. Further, the X-ray detector 105 is adapted to generate an electrical signal representative of the intensity distribution thereof. By moving the swing arm 101, the X-ray tube 104 and the detector 105 can be placed at any desired location and orientation relative to the patient 107.

The C-arm system 100 further comprises a control unit 155 and a data processing device 160, which are both accommodated within a workstation or a personal computer 150. The control unit 155 is adapted to control the operation of the C-arm system 100. The data processing device 160 is adapted for collecting 2D projection images of the object 107 for the purpose of reconstructing a 3D representation of the object 107. Further, the data processing device 160 is adapted to carry out the method for spatially characterizing a structure located within the object 107. The method will be described in more detail below.

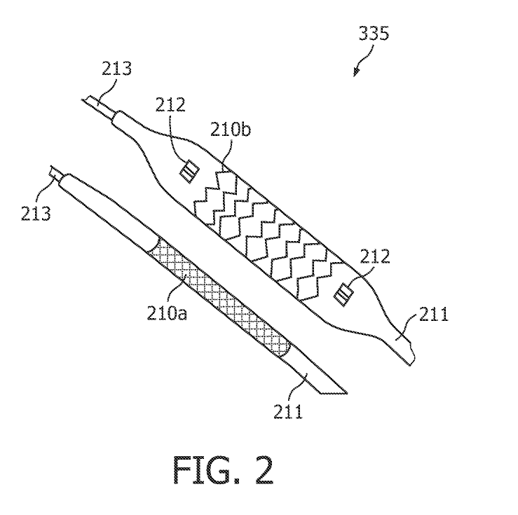

FIG. 2 shows an illustration of two stents for demonstrating the stent deployment, which, when being carried out within the body of a patient, cannot be seen in real. In the lower left part of FIG. 2 there is illustrated a stent 210a, which is existent in the initial state before an expansion is accomplished. The stent 210a is coupled to a delivery catheter 211, which is used to slide the stent 210a through a vessel tree in order to transport the stent 210a to a predefined location within the vascular system. Thereby, it is known to use a guide wire 213 in order to visualize the vessel path leading to the predefined location within the vascular system. Typically, the predefined location represents dangerous a narrowing or a stenosis within the vessel.

In order to perform a widening of the predefined vessel section the pressure within a balloon, which balloon is located inside the stent 210a, is increased. Thereby, the stent 210a will be deployed. Such a deployment procedure finally leads to a configuration as depicted in the upper right part of FIG. 2 showing a fully deployed stent 210b. In order to allow for an identification of the stent in X-ray images or in images being provided by other image modalities, the stents 210b is equipped with two reference markers 212.

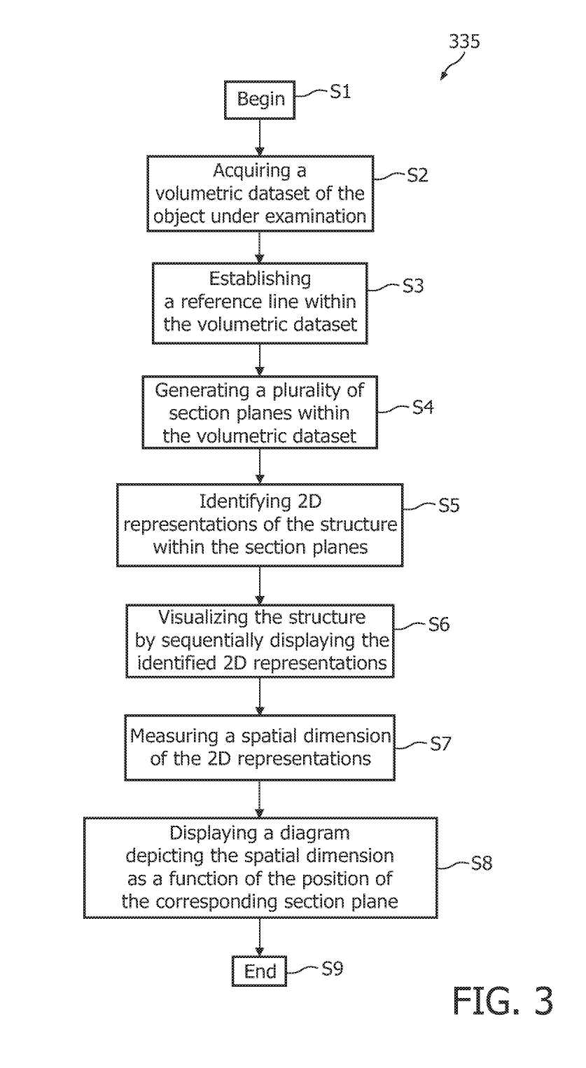

FIG. 3 shows a flow chart 335 on an exemplary method for spatially characterizing and visualizing a structure being located within an object under examination. The described method starts with a step S1.

In step S2 there is acquired a volumetric dataset of the object under examination. This dataset can be acquired by different image modalities. According to the embodiment described here the volumetric dataset is an X-ray attenuation dataset, which has been acquired by means of a C-arm system comprising an X-ray scanning unit being capable of rotating around the object under examination. The C-arm system further comprises a reconstruction unit for generating the volumetric dataset based on a plurality of different 2D X-ray projection data, which have been obtained at different viewing angles.

According to the embodiment described here, the object under examination is the heart of a patient. Since the human heart is a continuously moving object, the volumetric dataset may be generated be using a motion correction method, which has been described above in detail.

In step S3 there is established a reference line within the volumetric dataset. Preferably, the reference line is spatially located in such a manner that the reference line represents symmetry line of the object under examination or of the selected region of interest within the object under examination.

According to the embodiment described here, the reference line is approximately the center of a cardiac vessel. Therefore, reference markers being provided at a delivery catheter are used to define the 3D form and the 3D run of the reference line. In case the delivery catheter is used in connection with a guide wire, the guide wire itself representing a plurality of reference markers may be used to spatially define the reference line.

In step S4 there is generated a plurality of section planes within the volumetric dataset. Thereby, the section planes are oriented perpendicular with respect to the reference line. The section planes, which may also be denoted as cut planes, represent slices respectively slabs of the object under examination. The thickness of the slabs determines the spatial resolution of the described method in a direction aligned with the reference line.

In step S5 there is identified a 2D representation of the structure within each of the plurality of section planes. The identification of a 2D structure within the section plane may be carried out by applying known methods for image processing. Of course, for identifying the 2D structure pre-known properties of the 3D structure may be taken into account.

In step S6 there is carried out a visualization of the structure by displaying the identified 2D representations of the structure in a sequential manner. Thereby, the sequence of the displayed 2D representations corresponds to a section plane, which is continuously moving along the reference line.

At the beginning of the reference line a first section plane perpendicular to the reference line is defined or a slab of a certain thickness T is extracted and displayed. Along the reference line small steps of size S are made and each time a new section plane is displayed. This is done until the end of the reference line is reached.

Such a type of visualization corresponds to a virtual pullback, which is characterized by basically moving a section plane along the reference line that goes through the inside of the structure. The section planes being oriented perpendicular to the reference line are visualized as the position along the reference line changes. These views are in particular very familiar for the interventional cardiologists as they resemble like IVUS data and show a section plane through a vessel. However, a stent being inserted into the vessel can be visualized without having the hassles being related to known IVUS procedures.

In step S7 there is measured at least one spatial dimension of each of the identified 2D representations. The measurement of spatial dimensions of the 2D representations of the structure may be carried out in an automated manner by applying known methods for image processing. This allows for an automated quantitative characterization of the structure. This makes the described method even more reliable because a physician can be provided with absolute values regarding the size of the structure. Absolute values of the structure can be compared to e.g. standard values of a predefined range of values, which range is assigned to a known structure.

In step S8 there is displayed a diagram depicting the spatial dimension as a function of the position of the corresponding section plane with respect to the reference line. In case the structure is a stent or vessel lumen this may allow to plot for instance the diameter of the cylindrical element versus the position along the center axis of the cylindrical element. Thereby, a quantitative information about the cylindrical element may be given in a manner, which allows a physician to recognize the spatial characteristic of the structure very quickly and very easily.

Finally, the described exemplary method ends with step S9.

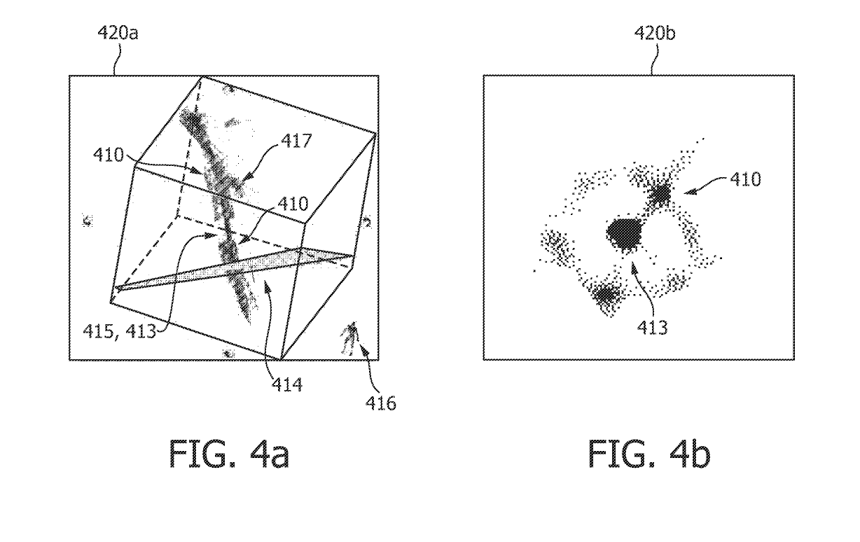

FIG. 4a shows an image 420a depicting two stents 410 and a guide wire 413, which all have been inserted into a portion of a vascular system. The guide wire 413 represents a reference line 415 for a plurality of different section planes 414 being oriented perpendicular to the reference line 415.

Further, in FIG. 4a there can been seen a tissue material 417, which is located in close proximity to a vessel portion of the vascular system and which has also been made visible by carrying out the above described method for spatially characterizing and visualizing a structure being located within a patient's body. At this point it has to be mentioned that certain types of human tissue respectively certain types of plaque can be seen in the reconstruction. The possibility to see and identify human tissue is a very exciting feature both for researchers and cardiologists.

In the lower right corner of the image 420a, there is depicted an insert 416, which directly gives an impression of the viewing direction of the image 420a with respect to the body of the patient. Further, there can be seen a section plane 414 virtually cutting the lower stent 410. This section plane 414 corresponds to a longitudinal position of a sectional view 420b of the stent 410 and the guide wire 413. This sectional view 420b is depicted in FIG. 4b. The sectional view 420b represents a perpendicular frame of both the stent 410 and the guide wire 413. The guide wire 413 can be identified in the center of the image 420b; the stent 410 is surrounding the guide wire 413 in a predominantly circular manner.

From a quantitative analysis of the image 420b, the following parameters corresponding to an exemplary embodiment of the described invention can be extracted:
a) Maximum diameter=2.4 mm
b) Minimum diameter=1.9 mm
c) Surface area=3.9 mm² (this corresponds to 91% of the maximum surface)
d) 87% of desired expansion
e) 75% of maximum expansion.

In this respect it has to be pointed out that the stent 410 is not perfectly circular. Therefore two diameters can be used to characterize the shape of the stent 410 in a more realistic manner. The maximum diameter and the minimum diameter.

FIG. 4c shows an image 420c depicting a perspective volumetric representation of a vessel lumen 419, which has been visualized by carrying out the above described method for spatially characterizing and visualizing a structure being located within a patient's body. Of course, not the vessel itself can be seen. What can be seen is the vessel lumen, which has been filled with an appropriate contrast agent being inserted into the vascular system of the patient under examination.

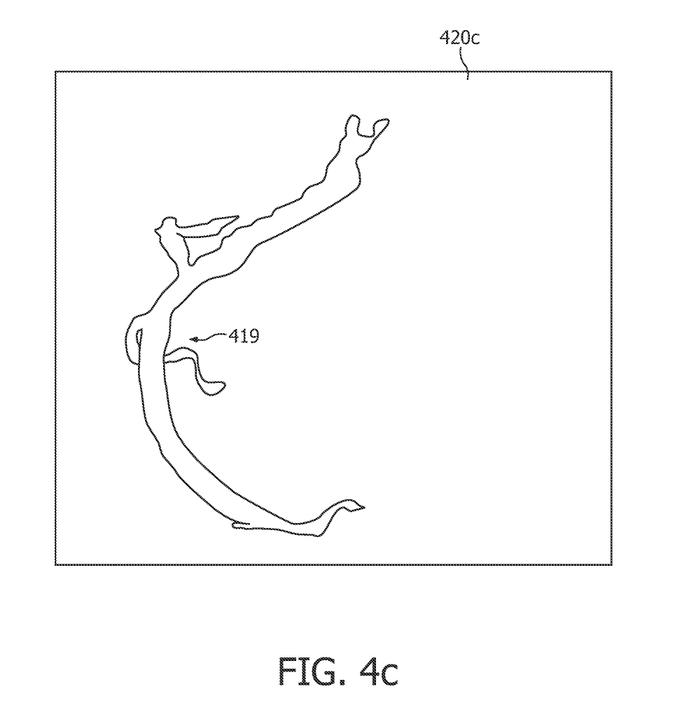

FIG. 5 shows a workflow diagram 530 for controlling a proper stent deployment. As indicated with reference numeral 531, the stent deployment starts with placing the stent with a predefined section of a narrowed vessel.

After having properly placed the up to now undeployed stent, rotational X-ray acquisitions are carried out which produce a plurality of different 2D projection dataset of the object under examination. These 2D projection datasets are combined by means of known reconstruction procedures in order to generate a 3D volumetric dataset of the object including the stent located within the object. The rotational acquisitions and the subsequent 3D reconstruction is indicated with reference numeral 532.

After having finished the 3D reconstruction a visual and a quantitative analysis of the stent and the corresponding vessel region is accomplished by carrying out a virtual pullback method as indicated with reference numeral 535. The virtual pullback method corresponds to the method, which has been explained in detail above with reference to FIG. 3. The quantitative analysis of the stent yields characteristic parameters, which describe the deployment state of the stent.

As indicated with reference numeral 536, the deployment state respectively the corresponding characteristic parameters are compared with set values.

If the final deployment state of the stent has not yet been reached, a reinflation of the stent or another appropriate procedure is carried out in order to amend the deployment state this is indicated with reference numeral 537. Thereafter, the above-described steps 532, 535 and 536 are repeated.

If step 536 shows, that the final deployment state of the stent has been reached, the stent deployment has been completed successfully. If step 536 shows, that the final deployment state of the stent has still not been reached, the above-described steps 537, 532, 535 and 536 are again repeated. This loop is carried out so often, until the final deployment state of the stent has been reached.

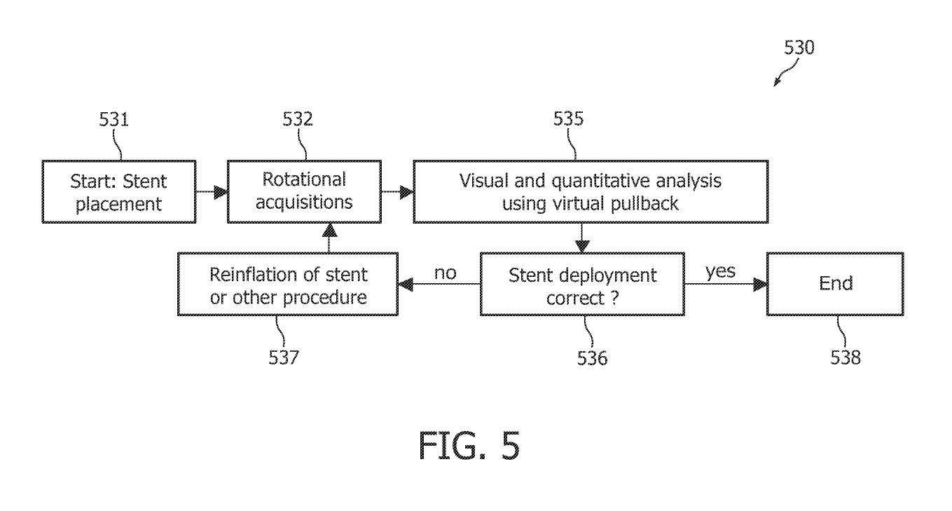

FIG. 6a shows a diagram 640 depicting the stent diameter d as a function of the longitudinal position p within the stent. The reference line p1 indicates the current position of a visualized section plane. The dotted curve 641 indicates the maximal stent diameter. The maximal stent parameter may be derived from specification parameters, which are typically given by the manufacturer of the stent.

The dotted curve 642 indicates the desired vessel lumen diameter, which should allow for a sufficient blood flow through the stent respectively through the vessel. The full line 643 indicates the actual maximum diameter and the full line 644 indicates the actual minimum diameter of an at least partially deployed stent. As has been described above, the actual maximum diameter and the actual minimum diameter allow for a characterization of the non-circularity of the stent.

FIG. 6b shows a diagram 645 depicting the stent cross-sectional area A as a function of the longitudinal position p within the stent. Again, the reference line p1 indicates the current position of a visualized section plane. The dotted curve 646 indicates the maximal allowable stent cross sectional area. This stent parameter may also be derived from specification parameters being provided by the manufacturer of the stent.

The dotted curve 647 indicates the desired stent cross sectional area after a perfect deployment of the stent. The full line 648 indicates the actual stent cross sectional area at a given longitudinal position p of the stent.

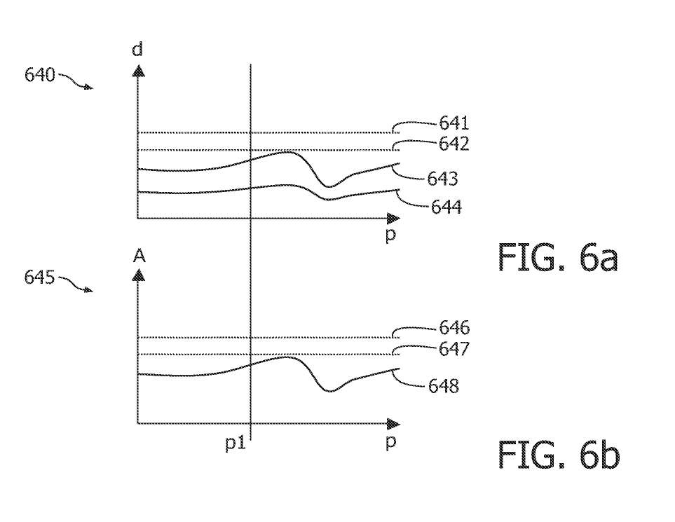

FIG. 7 shows a 3D visualization 770 of a deployed stent based on spatial measurements carried out by applying the above described method. The stent visualization 770 comprises different slabs of the stent, wherein the size of each slab has been calculated by means of the above-described quantitative analysis. As can be seen from FIG. 7, the stent 770 comprises a first portion 771, a second portion 772 and a third portion 773. The first portion 771 and the third portion 773 exhibit a correct stent expansion. The second portion exhibits a necking indicating that within the second portion 772 the stent is not deployed correctly.

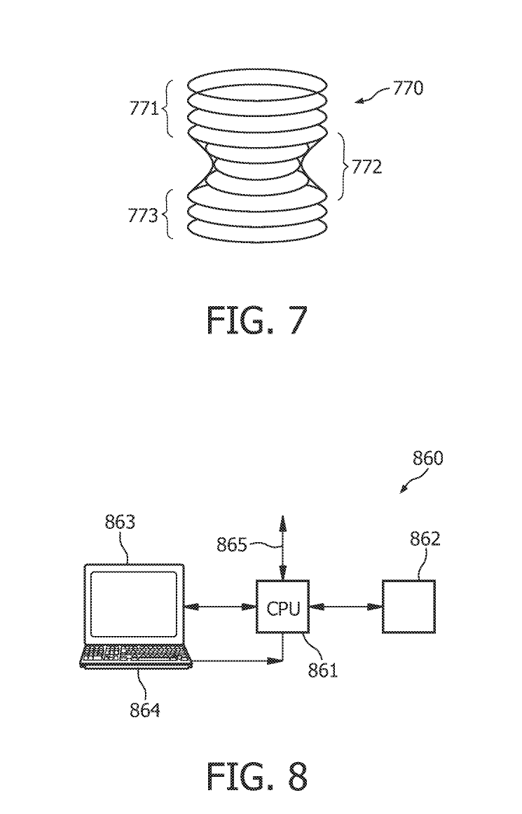

FIG. 8 shows an exemplary embodiment of a data processing device 860 according to the present invention for executing an exemplary embodiment of a method in accordance with the present invention. The data processing device 860 comprises a central processing unit or image processor 861. The image processor 861 is connected to a memory 862 for temporally storing acquired or processed datasets. Via a bus system 865 the image processor 861 is connected to a plurality of input/output network or diagnosis devices, such as a CT scanner and/or a C-arm being used e.g. for 3D rotational angiography. Furthermore, the image processor 861 is connected to a display device 863, for example a computer monitor, for visualizing the structure by displaying the identified 2D representations of the structure in a sequential manner. Further, the computer monitor may also be used for displaying a diagram depicting a spatial dimension of a structure being located within the object under examination as a function of the position of the corresponding section plane. An operator or user may interact with the image processor 861 via a keyboard 864 and/or via any other input/output devices.

It should be noted that the term “comprising” does not exclude other elements or steps and the “a” or “an” does not exclude a plurality. Also elements described in association with different embodiments may be combined. It should also be noted that reference signs in the claims should not be construed as limiting the scope of the claims.

In order to recapitulate the above described embodiments of the present invention one can state:

It is described a virtual pullback as a visualization and quantification tool that allows an interventional cardiologist to easily assess stent expansion. The virtual pullback visualizes the stent and/or the vessel lumen similar to an Intravascular Ultrasound (IVUS) pullback. The virtual pullback is performed in volumetric data along a reference line. The volumetric data can be a reconstruction of rotational 2D X-ray attenuation data. Planes perpendicular to the reference line are visualized as the position along the reference line changes. This view is for interventional cardiologists a very familiar view as they resemble IVUS data and may show a section plane through a vessel lumen or a stent. In these perpendicular section planes automatic measurements, such as minimum and maximum diameter, and cross sectional area of the stent can be calculated and displayed. Combining these 2D measurements allows also volumetric measurements to be calculated and displayed.

### LIST OF REFERENCE SIGNS

100 medical X-ray imaging system/C-arm system
101 swing arm scanning system/C-arm
102 patient table
103 robotic arm
104 X-ray tube
105 X-ray detector
106 X-ray
107 object under examination/patient
150 workstation/personal computer
155 control unit
160 data processing device
210a stent (before expansion)
210b stent (after expansion)
211 delivery catheter
212 reference marker
213 guide wire
335 flowchart for a visual and quantitative analysis using virtual pullback
S1 step 1
S2 step 2
S3 step 3
S4 step 4
S5 step 5
S6 step 6
S7 step 7
S8 step 8
S9 step 9
410 stent
413 guide wire
414 section plane
415 reference line
416 insert indicating the orientation of the depicted image
417 tissue
419 portion of a vascular system/vessel lumen
420a image depicting perspective volumetric representation of two stents and guide wire
420b image depicting perpendicular frame of the stent 410 and the guide wire 413
420c image depicting perspective volumetric representation of a vessel lumen
530 workflow diagram
531 stent placement
532 rotational X-ray acquisitions
535 visual and quantitative analysis using virtual pullback
536 check for correct stent deployment
537 reinflation of stent or other procedure
538 end
640 diagram depicting stent diameter
641 maximal stent diameter
642 desired vessel lumen diameter
643 actual maximum stent diameter
644 actual minimum stent diameter
645 diagram depicting stent cross sectional area
646 maximal stent cross sectional area
647 desired stent cross sectional area
648 actual stent cross sectional are
p position of section plane along stent
p1 position of current section plane
d diameter
A cross sectional area
770 3D visualization of expanded stent
771 first portion with correct expansion
772 second portion with incorrect expansion
773 third portion with correct expansion
860 data processing device
861 central processing unit/image processor
862 memory
863 display device
864 keyboard
865 bus system

1. A method for spatially characterizing a structure (210a, 210b, 410) located within an object under examination (107), in particular for spatially characterizing a medical device (210a, 210b, 410) being inserted into the body (107) of a patient, the method comprising the steps of
acquiring a volumetric dataset of the object under examination (107),
establishing a reference line (415) within the volumetric dataset,
generating a plurality of section planes (414) within the volumetric dataset, wherein the section planes (414) are oriented at least approximately perpendicular to the reference line (415), and
identifying 2D representations (420b) of the structure (210a, 210b, 410) within the plurality of section planes (414).

2. The method according to claim 1, wherein
the volumetric dataset represents the X-ray attenuation behavior of the object under examination (107).

3. The method according to claim 1, wherein
the volumetric dataset is a motion compensated dataset.

4. The method according to claim 1, wherein
the reference line (415) is defined by at least two reference markers (212) being inserted into the object under examination (107).

5. The method according to claim 1, wherein
the reference line (415) is defined by a guide wire (413).

6. The method according to claim 1, further comprising the step of
visualizing the structure (210a, 210b, 410) by displaying the identified 2D representations (420b) of the structure (210a, 210b, 410) in a sequential manner.

7. The method according to claim 1, further comprising the step of
displaying a 3D model representation (770) of the structure (210a, 210b, 410) by combining a plurality of identified 2D representations (420b) being assigned to a plurality of different section planes (414).

8. The method according to claim 1, further comprising the step of
measuring at least one spatial dimension of at least some of the identified 2D representations (420b).

9. The method according to claim 8, further comprising the step of
combining the at least one spatial dimension being measured for different 2D representations (420b) in such a manner that a 3D model representation (770) of the structure is established.

10. The method according to claim 8, wherein
the spatial dimension is the diameter (d) of the structure (210a, 210b, 410) and/or the cross sectional area (A) of the structure (210a, 210b, 410).

11. The method according to claim 8, further comprising the step of
displaying a diagram (640, 645) depicting the at least one spatial dimension as a function of the position of the corresponding section plane (414) with respect to the reference line (415).

12. The method according to claim 1, wherein
the reference line (415) is located within a vessel lumen (419) of a patient.

13. The method according to claim 12, wherein
the structure is a predetermined region of a vessel tree (419).

14. The method according to claim 13, wherein
at least one known property of a vessel lumen (419) is used in order to identify the vessel lumen (419) within the 2D representations.

15. The method according to claim 12, wherein
the structure is a stent (210a, 210b, 410).

16. The method according to claim 15, wherein
at least one known property of the stent (210a, 210b, 410) is used in order to identify the stent (210a, 210b, 410) within the 2D representations (420b).

17. The method according to claim 13, wherein
the step of acquiring a volumetric dataset of the object under examination (107) is carried out with contrast agent being inserted into the vessel lumen (419).

18. The method according to claim 17, further comprising the step of
acquiring a further volumetric dataset of the object under examination (107) in the absence of a contrast agent.

19. A data processing device
for spatially characterizing a structure (210a, 210b, 410) located within an object under examination (107), in particular for spatially characterizing a medical device (210a, 210b, 410) being inserted into the body (107) of a patient,
the data processing device (860) comprising
a data processor (861), which is adapted for performing the method as set forth in claim 1, and
a memory (862)
for storing the acquired volumetric dataset of the object under examination (107) and/or
for storing the identified 2D representation (420b) of the structure (210a, 210b, 410) and/or
for storing a movie of all or a selection of the identified 2D representations (420b) of the structure.

20. A medical X-ray examination apparatus, in particular a C-arm system (100) or a computed tomography system, the medical X-ray examination apparatus comprising
a data processing device (860) according to claim 19.

21. A computer-readable medium on which there is stored a computer program
for spatially characterizing a structure (210a, 210b, 410) located within an object under examination (107), in particular for spatially characterizing a medical device (210a, 210b, 410) being inserted into the body (107) of a patient,
the computer program, when being executed by a data processor (861), is adapted for controlling the method as set forth in claim 1.

22. A program element
for spatially characterizing a structure (210a, 210b, 410) located within an object under examination (107), in particular for spatially characterizing a medical device (210a, 210b, 410) being inserted into the body (107) of a patient,
the program element, when being executed by a data processor (861), is adapted for controlling the method as set forth in claim 1.
